(19) United States
(12) Patent Application Publication
(10) Pub. No.: US 2024/0216313 A1
(43) Pub. Date: Jul. 4, 2024

(54) ELAFIBRANOR DERIVATIVES AGONISTS OF PPAR FOR USE IN THE TREATMENT OF SEPSIS

(71) Applicant: Genfit, Loos (FR)

(72) Inventors: Vanessa Legry, Emmerin (FR); Remy Hanf, Lille (FR); Simon Debaecker, Pont-A-Marcq (FR); Philippe Poulain, Lorgies (FR); Benoit Noel, Gondecourt (FR); Robert Walczak, Lille (FR); Peggy Parroche, Loos (FR)

(21) Appl. No.: 18/289,213

(22) PCT Filed: May 10, 2022
(86) PCT No.: PCT/EP2022/062710

§ 371 Date: Nov. 1, 2023

(30) Foreign Application Priority Data
May 11, 2021 (EP) 21305615.3

(51) Int. Cl. A61K 31/192; A61K 31/407; A61P 31/00

(52) U.S. Cl. CPC A61K 31/192; A61K 31/407; A61P 31/00

(57) ABSTRACT

The invention relates to compounds for use in the treatment of sepsis.

The present invention is in the medical field and relates to compounds for use in the treatment of sepsis.

### BACKGROUND OF THE INVENTION

Sepsis is a dysregulated immune response to an infection that leads to organ dysfunction. It develops as the result of a complex, dysregulated host response to infection, a bacterial infection in most cases. This dysregulated host response is characterized not only by increased inflammation but also by immune suppression. The effects of this inappropriate response to infection lead to cellular dysfunction and, ultimately, organ failure. Single organ dysfunction in sepsis is rare, and several organs are usually affected. Mortality in patients with sepsis correlates with the number of organs that are affected.

Many patients with sepsis develop circulatory failure that results in abnormal cellular oxygen metabolism. Abnormal cellular oxygen metabolism manifests as an increase in blood lactate levels, typically to values >2 mEq per liter. Patients who require vasopressors to maintain a minimum mean arterial pressure despite adequate volume resuscitation and who have raised blood lactate levels are clinically diagnosed as having septic shock.

Current treatment for sepsis aims to limit the development of organ dysfunction by providing rapid control of infection, haemodynamic stabilization and organ support when possible to ensure recovery of organ function. But treatment of sepsis and septic shock remains a substantial unmet medical need.

### SUMMARY OF THE INVENTION

The present invention relates to a PPAR agonist selected from selected from lanifibranor, bezafibrate, fenofibrate, pemafibrate, seladelpar, saroglitazar, pioglitazone, rosiglitazone and a compound of formula (I) as defined below, or a pharmaceutically acceptable salt of a compound of formula (I), for use in a method for the treatment of sepsis in a subject in need thereof. In a particular embodiment, the invention relates to a PPAR agonist selected from lanifibranor, bezafibrate, pemafibrate, seladelpar, saroglitazar, pioglitazone, rosiglitazone and a compound of formula (I) as defined below, or a pharmaceutically acceptable salt of a compound of formula (I), for use in a method for the treatment of liver failure in a subject in need thereof.

In a particular embodiment, the PPAR agonist is selected from the following compounds, or pharmaceutically acceptable salt thereof:
2-[4-(3-methoxy-3-(4-(trifluoromethoxy)phenyl)propyl)-2,6-dimethylphenoxy]-2-methylpropanoic acid (cpd. 1);
2-[2,6-dimethyl-4-[3-[4-(trifluoromethyloxy)phenyl]-3-oxo-propyl]phenoxy]-2-methylpropanoic acid (cpd. 2);
2-[2,6-dimethyl-4-[3-[4-(trifluoromethyl)phenyl]-3-oxo-propyl]phenoxy]-2-methylpropanoic acid (cpd. 3);
2-[2,6-dimethyl-4-[3-[4-(methylthio)phenyl]-3-methoxypropyl]phenoxy]-2-methylpropanoic acid (cpd. 4);
2-[2,6-dimethyl-4-[3-[4-(methylthio)phenyl]-3-isopropyloxypropyl]phenoxy]-2-methylpropanoic acid (cpd. 5);
2-(4-(3-hydroxy-3-(4-(trifluoromethoxy)phenyl)propyl)-2,6-dimethylphenoxy)-2-methylpropanoic acid (cpd. 6);
2-(4-(3-(methoxyimino)-3-(4-(trifluoromethoxy)phenyl)propyl)-2,6-dimethylphenoxy)-2-methylpropanoic acid (cpd. 7);
2-(2-chloro-4-(3-(4-methyl-2-(4-(trifluoromethyl)phenyl)-thiazol-5-yl)-3-oxopropyl)phenoxy)-2-methylpropanoic acid (cpd. 8);
2-(2,3-dichloro-4-(3-ethoxy-3-(4-methyl-2-(4-(trifluoromethyl)-phenyl)thiazol-5-yl)propyl)phenoxy)-2-methylpropanoic acid (cpd. 9);
2-(4-(3-(benzyloxy)-3-(5-(4-(trifluoromethyl)phenyl)thien-2-yl)propyl)-2,3-dichlorophenoxy)-2-methylpropanoic acid (cpd. 10);
2-(2,3-dichloro-4-(3-methoxy-3-(5-(4-(trifluoromethyl)phenyl)-thien-2-yl)propyl)phenoxy)-2-methylpropanoic acid (cpd. 11);
2-[2,6-dimethyl-4-[3-[4-(methylthio)phenyl]-3-methoxypropyl]phenoxy]-2-methylpropanoic acid (cpd. 13); and
2-[2,6-dimethyl-4-[3-[4-bromophenyl]-3-methoxypropyl]phenoxy]-2-methylpropanoic acid (cpd. 15);
   or a pharmaceutically acceptable salt thereof.

In a further particular embodiment, the compound is cpd. 1 or a pharmaceutically acceptable salt thereof.

In a particular embodiment, the PPAR agonist is administered to a subject who suffers from or is at risk of sepsis with multiple organ failure. In another embodiment, the subject suffers from or is at risk of septic shock.

In another embodiment, the PPAR agonist is for use as a single active agent in said method.

In yet another embodiment, the PPAR agonist is for use in combination with an antimicrobial agent in the method disclosed herein. In another embodiment, the antimicrobial agent is an antibiotic, in particular a carbapenem antibiotic, such as ertapenem.

### DESCRIPTION OF THE FIGURES

FIG. 1: Compounds according to the invention reduce TNFα and MCP1 secretion in PMA-stimulated THP1 monocytes.

FIGS. 1A and 1B show the effect of Cpd.1 on the reduction of TNFα and MCP1 secretion respectively in PMA-stimulated THP1.

For FIGS. 1A and 1B: #, ##, ### for p<0.05, p<0.01, p<0.001 compared to the vehicle (Veh) using ANOVA and Fisher's LSD test for multiple comparisons; *, **, *** for p<0.05, p<0.01, p<0.001 compared to the vehicle (Veh) using non-parametric Kruskall Wallis test with uncorrected Dunn's test fir multiple comparisons. PMA dose: 100 ng/mL.

FIG. 2: Compounds according to the invention reduce cytokine production by THP1 differentiated macrophages.

FIG. 2A shows the effect of Cpd.1 on the reduction of TNFα production by THP1 differentiated macrophages. # for p<0.05 using non-parametric Dunn's test for multiple comparison between Cpd.1 and the vehicle (Veh).

FIG. 2B shows the effect of Cpd.1 on the reduction of MCP1 production by THP1 differentiated macrophages. ### for p<0.001 using non-parametric Dunn's test for multiple comparison between Cpd.3 and the vehicle (Veh).

FIG. 3: Reduction of serum cytokine concentration in response to LPS in rats.

FIGS. 3A and 3B shows the effect of Cpd.1 on the reduction of serum IL6 and IL1β concentration respectively in response to LPS in rats.

#, ##, ### for p<0.05, p<0.01, p<0.001 using Student T test.$ for p<0.05 using non-parametric Mann-Whitney test.

FIG. 4: Effect of Cpd.1 and Cpd.19 on hepatic function and cytokine level in a model of endotoxemia.

Rats were treated with Cpd.1 (3 mg/kg), Cpd.19 (100 mg/kg) or a vehicle (Veh.) every day for 3 days before LPS injection. Blood was collected 3 h after LPS injection for the measurement of total bilirubin (A), serum albumin (B) and TNFα (C) in the serum. For A-B, One-way Anova with Dunnett test for multiple testing was used to assess statistical significance. For C, One-way Anova was used to assess statistical significance. *** p<0.001, *p<0.05 FIG. 5: Effect of Cpd.1 on survival rate in a model of sepsis.

Cecal ligation and puncture surgery (CLP) was performed in mice at 0 h. Cpd.1 or vehicle was administrated at 0.3 mg/kg, p.o. for three days before CLP surgery and the mice were monitored for survival during 7 days (168 days). Mice found dead in the morning are counted with those from the afternoon of the day before. Statistical difference between the experimental groups was determined by using Gehan-Breslow-Wilcoxon test. *p<0.0332

FIG. 6: Effect of Cpd. on MCP1 secretion induced by LPS in THP1 macrophages.

After differentiation into macrophages, THP1 cells were treated for 24 h with 1 or 10 μM of indicated Cpd. before stimulation for 6 h with LPS from Klebsiella. The % inhibition of MCP1 secretion was calculated over the mean LPS-vehicle condition (Veh.). Student t-test was used to assess statistical significance. Grey boxes depict significant values (p<0.05).

FIG. 7: Effect of Cpd. on TNFα secretion induced by LPS in THP1 macrophages.

After differentiation into macrophages, THP1 cells were treated for 24 h with 1 or 10 μM of the indicated Cpd. before stimulation for 6 h with LPS from Klebsiella. The % inhibition of TNFα secretion was calculated over the mean LPS-vehicle condition (Veh.). Student t-test was used to assess statistical significance. Grey boxes depict significant values (p<0.05).

FIG. 8: Effect of Cpd. on staurosporin-induced apoptosis in HepG2 cells.

HepG2 cells were pre-treated with the indicated Cpd. at 0.3 μM to 10 μM for 16 h before incubation of 10 μM staurosporin for additional 4 hours. Apoptosis was assessed through caspase 3/7 activity measurement. The % inhibition of caspase 3/7 activity was calculated over the mean staurosporin-vehicle condition (Veh.). Student t-test was used to assess statistical significance. Grey boxes depict significant values (p<0.05).

### DETAILED DESCRIPTION OF THE INVENTION

The present invention relates to a PPAR agonist selected from lanifibranor, bezafibrate, fenofibrate, pemafibrate, seladelpar, saroglitazar, pioglitazone, rosiglitazone and a compound of formula (I) as defined below, or a pharmaceutically acceptable salt of a compound of formula (I), for use in the treatment of sepsis.

### Definitions

In the context of the present invention, the terms below have the following meanings.

The terms mentioned herein with prefixes such as for example C1-C6, can also be used with lower numbers of carbon atoms such as C1-C2. If, for example, the term C1-C6 is used, it means that the corresponding hydrocarbon chain may comprise from 1 to 6 carbon atoms, especially 1, 2, 3, 4, 5, or 6 carbon atoms. If, for example, the term C1-C3 is used, it means that the corresponding hydrocarbon chain may comprise from 1 to 3 carbon atoms, especially 1, 2, or 3 carbon atoms.

The term “alkyl” refers to a saturated, linear or branched aliphatic group. The term “(C1-C6)alkyl” more specifically means methyl, ethyl, propyl, isopropyl, butyl, pentyl, or hexyl. In a preferred embodiment, the “alkyl” is a methyl.

The term “alkoxy” or “alkyloxy” corresponds to the alkyl group as above defined bonded to the molecule by an —O— (ether) bond. (C1-C6)alkoxy includes methoxy, ethoxy, propyloxy, isopropyloxy, butyloxy, pentyloxy, or hexyloxy. In a preferred embodiment, the “alkoxy” or “alkyloxy” is a methoxy, an ethoxy, a propoxy, an isopropyloxy, more preferably a methoxy.

The term “alkylthio” corresponds to the alkyl group as above defined bonded to the molecule by an —S— (thioether) bond. (C1-C6)alkylthio includes thiomethyl, thioethyl, thiopropyl, thioisopropyl, thiobutyl, thiopentyl, or thiohexyl. In a preferred embodiment, the “alkylthio” is a thiomethyl, a thioethyl, a thiopropyl, a thioisopropyl, more preferably a thiomethyl.

A “cyclic” group corresponds to an aryl group, a cycloalkyl group or a heterocyclic group.

The term “aryl” corresponds to a mono- or bi-cyclic aromatic hydrocarbons having from 6 to 12 carbon atoms. For instance, the term “aryl” includes phenyl, naphthyl, or anthracenyl. In a preferred embodiment, the aryl is a phenyl.

The term “cycloalkyl” corresponds to a saturated or unsaturated mono-, bi- or tri-cyclic alkyl group comprising between 3 and 20 atoms of carbons. It also includes fused, bridged, or spiro-connected cycloalkyl groups. The term “cycloalkyl” includes for instance cyclopropyl, cyclobutyl, cyclopentyl, and cyclohexyl, preferably cyclopropyl. The term “spirocycloalkyl” includes for instance a spirocyclopropyl.

The term “cycloalkoxy” corresponds to the cycloalkyl group as above defined bonded to the molecule by an —O— (ether) bond.

The term “cycloalkylthio” corresponds to the cycloalkyl group as above defined bonded to the molecule by an —S— (thioether) bond.

The term “heterocycloalkyl” corresponds to a saturated or unsaturated cycloalkyl group as above defined further comprising at least one heteroatom such as nitrogen, oxygen, or sulphur atom, preferably at least one nitrogen atom. It also includes fused, bridged, or spiro-connected heterocycloalkyl groups. Representative heterocycloalkyl groups include, but are not limited to dioxolanyl, benzo[1,3]dioxolyl, azetidinyl, oxetanyl, pyrazolinyl, pyranyl, thiomorpholinyl, pyrazolidinyl, piperidyl, piperazinyl, 1,4-dioxanyl, imidazolinyl, pyrrolinyl, pyrrolidinyl, piperidinyl, imidazolidinyl, morpholinyl, 1,4-dithianyl, pyrrolidinyl, oxozolinyl, oxazolidinyl, isoxazolinyl, isoxazolidinyl, dithiolanyl, azepanyl, thiazolinyl, thiazolidinyl, isothiazolinyl, isothiazolidinyl, dihydropyranyl, tetrahydropyranyl, tetrahydrofuranyl, and tetrahydrothiophenyl. In a preferred embodiment, the heterocycloalkyl group is morpholinyl, piperazinyl, piperidinyl, pyrrolidinyl, tetrahydropyranyl, dithiolanyl and azepanyl groups, more preferably piperidinyl.

The term “heteroaryl” as used herein corresponds to an aromatic, mono- or poly-cyclic group comprising between 5 and 14 atoms and comprising at least one heteroatom such as nitrogen, oxygen or sulphur atom. As used herein, the term “heteroaryl” further includes the “fused arylheterocycloalkyl” and “fused heteroarylcycloalkyl”. The terms “fused arylheterocycloalkyl” and “fused heteroarylcycloalkyl” correspond to a bicyclic group in which an aryl as above defined or a heteroaryl is respectively bounded to the heterocycloalkyl or the cycloalkyl as above defined by at least two carbons. In other terms, the aryl or the heteroaryl respectively shares a carbon bond with the heterocycloalkyl or the cycloalkyl. Examples of such mono- and poly-cyclic heteroaryl groups may be: pyridinyl, thiazolyl, thiophenyl, furanyl, pyrrolyl, pyrazolyl, imidazolyl, triazolyl, tetrazolyl, benzofuranyl, thianaphthalenyl, indolyl, indolinyl, quinolinyl, isoquinolinyl, benzimidazolyl, tetrahydroquinolinyl, tetrahydroisoquinolinyl, triazinyl, thianthrenyl, isobenzofuranyl, chromenyl, xanthenyl, phenoxanthinyl, isothiazolyl, isoxazolyl, pyrazinyl, pyridazinyl, indolizinyl, isoindolyl, indazolyl, purinyl, quinolizinyl, phtalazinyl, naphthyridinyl, quinoxalinyl, quinazolinyl, cinnolinyl, pteridinyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, indolinyl, isoindolinyl, oxazolidinyl, benzotriazolyl, benzoisoxazolyl, oxindolyl, benzoxazolyl, benzoxazolinyl, benzoxazinyl, benzothienyl, benzothiazolyl, benzodiazepinyl, benzazepinyl, benzoxazepinyl, isatinyl, dihydropyridyl, pyrimidinyl, s-triazinyl, oxazolyl, or thiofuranyl. In a preferred embodiment, a heteroaryl is a thiazolyl, pyridinyl, pyrimidinyl, furanyl, thiophenyl, quinolinyl, and isoquinolinyl, more preferably a thiazolyl and thiophenyl.

The term “heterocyclic” refers to a heterocycloalkyl group or a heteroaryl group as above defined.

The term “halogen” corresponds to a fluorine, chlorine, bromine, or iodine atom, preferably a fluorine atom, a chlorine atom or a bromine atom.

The term “pharmaceutically acceptable salts” includes inorganic as well as organic acids salts.

Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically acceptable inorganic or organic acid addition salts include the pharmaceutically acceptable salts listed in J. Pharm. Sci. 1977, 66, 2, and in Handbook of Pharmaceutical Salts: Properties, Selection, and Use edited by P. Heinrich Stahl and Camille G. Wermuth 2002. The “pharmaceutically acceptable salts” also include inorganic as well as organic base salts. Representative examples of suitable inorganic bases include sodium or potassium salt, an alkaline earth metal salt, such as a calcium or magnesium salt, or an ammonium salt. Representative examples of suitable salts with an organic base includes for instance a salt with methylamine, dimethylamine, trimethylamine, piperidine, morpholine or tris-(2-hydroxyethyl)amine.

As used herein, the terms “treatment”, “treat” or “treating” refer to any act intended to ameliorate the health status of patients such as therapy, prevention, prophylaxis and retardation of a disease. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease. In a particular embodiment, the invention is used to reduce the mortality associated to sepsis. In other embodiments, the invention can be used to slow or stop the progression of sepsis. In particular, the invention can be used to prevent the progression of sepsis, in particular to prevent the progression of sepsis to septic shock in a subject suffering from sepsis. In other embodiments, the invention can be used to prevent organ failure, in particular multiple organ failure, in a subject suffering from sepsis.

As used herein, the terms “subject”, “individual” or “patient” are interchangeable and refer to an animal, preferably to a mammal, even more preferably to a human, including adult, child, newborn and human at the prenatal stage. However, the term “subject” can also refer to non-human animals, in particular mammals such as dogs, cats, horses, cows, pigs, sheeps and non-human primates, among others.

The expression “substituted by at least” means that the radical is substituted by one or several groups of the list.

In the context of the present invention, the term “about” applied to a numerical value means the value +/−10%. For the sake of clarity, this means that “about 100” refers to values comprised in the 90-110 range. In addition, in the context of the present invention, the term “about X”, wherein X is a numerical value, also discloses specifically the X value, but also the lower and higher value of the range defined as such, more specifically the X value.

### Compounds for Use According to the Present Invention

The present invention provides a PPAR agonist selected from lanifibranor, bezafibrate, fenofibrate, pemafibrate, seladelpar, saroglitazar, pioglitazone, rosiglitazone and a compound of formula (I) as defined below, or a pharmaceutically acceptable salt of a compound of formula (I), for use in a method for the treatment of sepsis. In yet another particular embodiment, the invention provides a PPAR agonist selected from lanifibranor, bezafibrate, pemafibrate, seladelpar, saroglitazar, pioglitazone, rosiglitazone and a compound of formula (I) as defined below, or a pharmaceutically acceptable salt of a compound of formula (I), for use in a method for the treatment of liver failure.

In a particular embodiment, the PPAR agonist is selected from lanifibranor, bezafibrate, fenofibrate, pemafibrate, seladelpar, saroglitazar, pioglitazone and rosiglitazone. In a further particular embodiment, the PPAR agonist is selected from lanifibranor, bezafibrate, pemafibrate, seladelpar, saroglitazar, pioglitazone and rosiglitazone.

In another particular embodiment, the PPAR agonist for use according to the invention is a compound of formula (I), or a pharmaceutically acceptable salt thereof,

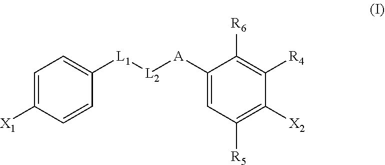

wherein:
X1 represents a halogen atom, a R1 group or a G1-R1 group;
L1 represents a bond, a thiophenyl group or a thiazole group substituted or not by a (C1-C3)alkyl group;
L2 represents:
   (i) a —CH—OR7 group, in which R7 represents a hydrogen atom, an unsubstituted (C1-C6)alkyl group or a (C1-C6)alkyl group substituted by a (C6-C14)aryl group, in particular in which R7 represents an unsubstituted (C1-C6)alkyl group or a (C1-C6)alkyl group substituted by a (C6-C14)aryl group;
   (ii) a carbonyl group (CO); or
   (iii) a C═N—OR8, in which R8 represents an unsubstituted (C1-C6)alkyl group;
A represents a CH═CH or a CH₂—CH₂ group;
X2 represents a G2-R2 group;
G1 and G2, identical or different, represent an atom of oxygen or sulfur;
R1 represents a hydrogen atom, an unsubstituted (C1-C6)alkyl group, a (C6-C14)aryl group or an alkyl group that is substituted by at least one substituent selected from halogen atoms, (C1-C6)alkoxy groups, (C1-C6)alkylthio groups, (C5-C10)cycloalkyl groups, (C5-C10)cycloalkylthio groups and 5- to 14-membered heterocyclic groups;
R2 represents a (C1-C6)alkyl group substituted by a —COOR3 group;
R3 represents a hydrogen atom or a (C1-C6)alkyl group that is substituted or not by at least one substituent selected from halogen atoms, (C5-C10)cycloalkyl groups and 5- to 14-membered heterocyclic groups;
R4 represents a halogen atom, an unsubstituted (C1-C6)alkyl group or a (C1-C6)alkyl group that is substituted by at least one substituent selected from halogen atoms, (C5-C10)cycloalkyl groups and 5- to 14-membered heterocyclic groups;
R5 represents a hydrogen atom, a halogen atom, an unsubstituted (C1-C6)alkyl group or a (C1-C6)alkyl group that is substituted by at least one substituent selected from halogen atoms, (C5-C10)cycloalkyl groups and 5- to 14-membered heterocyclic groups; and
R6 represents a hydrogen atom or a halogen atom;
with the proviso that the compound of formula (I) is not:
   elafibranor or a pharmaceutically acceptable salt thereof; or 2-[2,6-dimethyl-4-[3-[4-(methylthio)phenyl]-3-oxo-propyl]phenoxy]-2-methylpropanoic acid or a pharmaceutically acceptable salt thereof.

In a particular embodiment, L1 represents a bond, and R6 is a hydrogen atom, i.e. the compound of formula (I) is a compound of formula (Ia) as represented below:

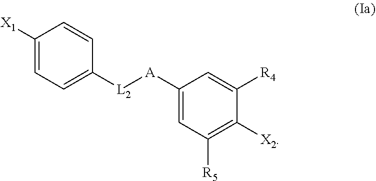

In another embodiment, L1 represents a thiazol group that is substituted or not by a (C1-C3)alkyl group, in particular by a methyl group. In a particular embodiment, L1 represents a 2-methyl-thiazolyl group. In a further particular embodiment, L1 is a 2-methyl-thiazolyl group and the compound of formula (I) is a compound of formula (Ib) as represent below:

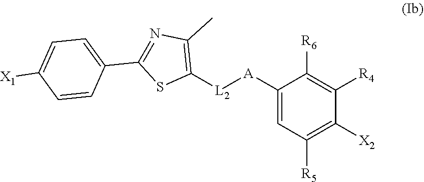

In another particular embodiment, L1 represents a thiophenyl group. In yet another embodiment, L1 represents a thiophenyl group and the compound of formula (I) is a compound of formula (Ic) as represented below:

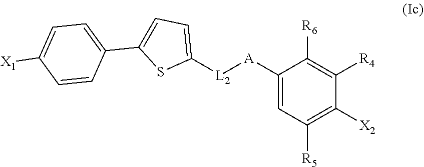

In a particular embodiment, X1 is a R1 group wherein R1 is an unsubstituted (C1-C6)alkyl group or a (C1-C6)alkyl group that is substituted by one or more halogen atoms.

In another particular embodiment, X1 is a R1 group wherein R1 is an unsubstituted (C1-C6)alkyl group. In another particular embodiment, X1 is a R1 group wherein R1 is an unsubstituted (C1-C4)alkyl group. In another particular embodiment, X1 is a R1 group wherein R1 is an unsubstituted (C1-C3)alkyl group. In another particular embodiment, X1 is a R1 group wherein R1 is a methyl or ethyl group. In another particular embodiment, X1 is a R1 group wherein R1 is a methyl group.

In another particular embodiment, X1 is a R1 group wherein R1 is a (C1-C6)alkyl group that is substituted by one or more halogen atoms. In another particular embodiment, X1 is a R1 group wherein R1 is a (C1-C4)alkyl group that is substituted by one or more halogen atoms. In another particular embodiment, X1 is a R1 group wherein R1 is a (C1-C3)alkyl group that is substituted by one or more halogen atoms. In another particular embodiment, X1 is a R1 group wherein R1 is a methyl or ethyl group substituted by one or more halogen atoms. In another particular embodiment, X1 is a R1 group wherein R1 is a methyl group substituted by one or more halogen atoms. In another particular embodiment, X1 is a R1 group wherein R1 is a trifluoromethyl group.

In a particular embodiment, G1 is a sulfur atom.

In another particular embodiment, G1 is a sulfur atom and R1 is a (C1-C6)alkyl group that is substituted by one or more halogen atoms. In another particular embodiment, G1 is a sulfur atom and R1 is a (C1-C4)alkyl group that is substituted by one or more halogen atoms. In another particular embodiment, G1 is a sulfur atom and R1 is a (C1-C3)alkyl group that is substituted by one or more halogen atoms. In another particular embodiment, G1 is a sulfur atom and R1 is a methyl or ethyl group substituted by one or more halogen atoms. In another particular embodiment, G1 is a sulfur atom and R1 is a methyl group substituted by one or more halogen atoms. In another particular embodiment, G1 is a sulfur atom and R1 is a trifluoromethyl group.

In a further particular embodiment, G1 is a sulfur atom and R1 is an unsubstituted (C1-C6)alkyl group or a (C1-C6)alkyl group that is substituted by one or more halogen atoms. In another particular embodiment, G1 is a sulfur atom and R1 is an unsubstituted (C1-C6)alkyl group. In another particular embodiment, G1 is a sulfur atom and R1 is an unsubstituted (C1-C4)alkyl group. In another particular embodiment, G1 is a sulfur atom and R1 is an unsubstituted (C1-C3)alkyl group. In another particular embodiment, G1 is a sulfur atom and R1 is a methyl or ethyl group. In another particular embodiment, G1 is a sulfur atom and R1 is a methyl group.

In yet another particular embodiment, G1 is an oxygen atom.

In a further particular embodiment, G1 is an oxygen atom and R1 is an unsubstituted (C1-C6)alkyl group or a (C1-C6)alkyl group that is substituted by one or more halogen atoms. In another particular embodiment, G1 is an oxygen atom and R1 is an unsubstituted (C1-C6)alkyl group. In another particular embodiment, G1 is an oxygen atom and R1 is an unsubstituted (C1-C4)alkyl group. In another particular embodiment, G1 is an oxygen atom and R1 is an unsubstituted (C1-C3)alkyl group. In another particular embodiment, G1 is an oxygen atom and R1 is a methyl or ethyl group. In another particular embodiment, G1 is an oxygen atom and R1 is a methyl group.

In another particular embodiment, G1 is an oxygen atom and R1 is a (C1-C6)alkyl group that is substituted by one or more halogen atoms. In another particular embodiment, G1 is an oxygen atom and R1 is a (C1-C4)alkyl group that is substituted by one or more halogen atoms.

In another particular embodiment, G1 is an oxygen atom and R1 is a (C1-C3)alkyl group that is substituted by one or more halogen atoms. In another particular embodiment, G1 is an oxygen atom and R1 is a methyl or ethyl group substituted by one or more halogen atoms. In another particular embodiment, G1 is an oxygen atom and R1 is a methyl group substituted by one or more halogen atoms. In another particular embodiment, G1 is an oxygen atom and R1 is a trifluoromethyl group.

In another particular embodiment, G2 is an oxygen atom.

In a further particular embodiment, R2 represents a (C1-C4)alkyl group that is substituted by a —COOR3 group. In another embodiment, R2 represents a (C1-C3)alkyl group that is substituted by a COOR3 group. In another embodiment, R2 represents a C(CH₃)₂ group substituted by a —COOR3 group.

In another particular embodiment, R3 is a hydrogen atom or an unsubstituted (C1-C6)alkyl group. In another embodiment, R3 is a hydrogen atom or an unsubstituted (C1-C4)alkyl group.

In another embodiment, R3 is a hydrogen atom or methyl, ethyl, propyl, isopropyl, butyl, n-butyl, isobutyl or tertbutyl group. In another particular embodiment, R3 is a hydrogen atom.

In another particular embodiment, R4 is a halogen atom or an unsubstituted (C1-C6)alkyl group. In another embodiment, R4 is a chlorine atom. In another embodiment, R4 is an unsubstituted (C1-C6)alkyl group. In another embodiment, R4 is an unsubstituted (C1-C4)alkyl group. In another embodiment, R4 is an unsubstituted (C1-C3)alkyl group. In another embodiment, R4 is a methyl or ethyl group. In another embodiment, R4 is a methyl group.

In another particular embodiment, R5 is hydrogen atom or an unsubstituted (C1-C6)alkyl group. In a particular embodiment, R5 is a hydrogen atom. In another embodiment, R5 is an unsubstituted (C1-C6)alkyl group. In another embodiment, R5 is an unsubstituted (C1-C4)alkyl group. In another embodiment, R5 is an unsubstituted (C1-C3)alkyl group. In another embodiment, R5 is a methyl or ethyl group. In another embodiment, R5 is a methyl group.

In another particular embodiment, R4 and R5 are identical. In another embodiment, R4 and R5 are an unsubstituted (C1-C6)alkyl group. In another embodiment, R4 and R5 are an unsubstituted (C1-C6)alkyl group. In another embodiment, R4 and R5 are an unsubstituted (C1-C4)alkyl group. In another embodiment, R4 and R5 are an unsubstituted (C1-C3)alkyl group. In another embodiment, R4 and R5 are a methyl or ethyl group. In another embodiment, R4 and R5 are a methyl group.

In a particular embodiment, R6 is a halogen atom. In another embodiment, R6 is a chlorine atom.

In another particular embodiment, R4 and R6 are identical. In another embodiment, R4 and R6 are a halogen atom. In another embodiment, R4 and R6 are chlorine atom.

In another particular embodiment, L2 is a —CH—OR7 group. In another embodiment, R7 is an unsubstituted (C1-C4)alkyl group. In another embodiment, R7 is an unsubstituted (C1-C3)alkyl group. In another embodiment, R7 is a methyl or ethyl group. In another embodiment, R7 is a methyl group.

In a particular embodiment, R7 is a (C1-C6)alkyl substituted by a phenyl group. In another particular embodiment, R7 is a methyl or ethyl group substituted by a phenyl group. In yet another embodiment, R7 is a benzyl group.

In another particular embodiment, L2 is a carbonyl group.

In another particular embodiment, L2 is a C═N—OR8. In another embodiment, R8 is an unsubstituted (C1-C4)alkyl group. In another embodiment, R8 is an unsubstituted (C1-C3)alkyl group. In another embodiment, R8 is a methyl or ethyl group. In another embodiment, R8 is a methyl group.

In another particular embodiment, the PPAR agonist is a compound of formula (Ia) wherein:
G1 is a sulfur atom;
R1 is an unsubstituted (C1-C4)alkyl group;
R2 is a (C1-C3)alkyl group substituted by a —COOR3 group;
R3 is a hydrogen atom or an unsubstituted (C1-C4)alkyl group;
R4 and R5 represent a (C1-C4)alkyl group; and
L2 is a carbonyl group.

In another particular embodiment, the PPAR agonist is a compound of formula (Ia) wherein:
G1 is a sulfur atom;
R1 is an unsubstituted (C1-C4)alkyl group;
R2 is a C(CH₃)₂ group substituted by a —COOR3 group;
R3 is a hydrogen atom or an unsubstituted (C1-C4)alkyl group;
R4 and R5 represent a (C1-C4)alkyl group; and
L2 is a carbonyl group.

In another particular embodiment, the PPAR agonist is a compound of formula (Ia) wherein:
G1 is a sulfur atom;
R1 is an unsubstituted (C1-C4)alkyl group;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a carbonyl group; and
A is a CH═CH group.

In another particular embodiment, the PPAR agonist is a compound of formula (Ia) wherein:
G1 is a sulfur atom;
R1 is an unsubstituted (C1-C4)alkyl group;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a carbonyl group; and
A is a CH₂—CH₂ group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a (C1-C3)alkyl group substituted by a —COOR3 group;
R3 is a hydrogen atom or an unsubstituted (C1-C4)alkyl group;
R4 and R5 represent a (C1-C4)alkyl group; and
L2 is a —CH—OR7 group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a (C1-C3)alkyl group substituted by a —COOR3 group;
R3 is a hydrogen atom or an unsubstituted (C1-C4)alkyl group;
R4 and R5 represent a (C1-C4)alkyl group;
L2 is a —CH—OR7 group; and
R7 is an unsubstituted (C1-C4)alkyl group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a —CH—OR7 group; and
R7 is an unsubstituted (C1-C4)alkyl group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a —CH—OR7 group;
R7 is an unsubstituted (C1-C4)alkyl group; and
A is a CH═CH group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a —CH—OR7 group;
R7 is an unsubstituted (C1-C4)alkyl group; and
A is a CH₂—CH₂ group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a —CH—OR7 group;
R7 is a (C1-C6)alkyl group substituted by a (C6-C14)aryl group; and
A is a CH═CH group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a (C1-C3)alkyl group substituted by a —COOR3 group;
R3 is a hydrogen atom or an unsubstituted (C1-C4)alkyl group;
R4 and R5 represent a (C1-C4)alkyl group;
L2 is a C═N—OR8 group; and
R8 represents an unsubstituted (C1-C6) alkyl group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a C═N—OR8 group; and
R8 is an unsubstituted (C1-C4)alkyl group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a C═N—OR8 group;
R8 is an unsubstituted (C1-C4)alkyl group; and
A is a CH═CH group.

In another particular embodiment, the PPAR agonist is a compound of formula (Ia) wherein:
G1 is an oxygen atom;
R1 is an unsubstituted (C1-C4)alkyl group or a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 and R5 represent a (C1-C4)alkyl group;
L2 is a C═N—OR8 group;
R8 is an unsubstituted (C1-C4)alkyl group; and
A is a CH₂—CH₂ group.

In another particular embodiment, the PPAR agonist is a compound of formula (Ib) wherein X1 is a R1 group. In another particular embodiment, the PPAR agonist is a compound of formula (Ib) wherein X1 is a R1 group wherein R1 is a (C1-C6)alkyl group substituted by at least one halogen atoms. In another particular embodiment, the PPAR agonist is a compound of formula (Ib) wherein X1 is a R1 group wherein R1 is a CF3 group.

In another particular embodiment, the PPAR agonist is a compound of formula (Ib) wherein L2 represents a —CH—OR7 group or a carbonyl group. In yet another embodiment, L2 represents a carbonyl group in formula (Ib).

In a further particular embodiment, the PPAR agonist is a compound of formula (Ib) wherein R4 is a halogen atom. In yet another embodiment, R4 is a chlorine atom in formula (Ib).

In a further particular embodiment, the PPAR agonist is a compound of formula (Ib) wherein R5 is a hydrogen atom.

In another embodiment, the PPAR agonist is a compound of formula (Ib) wherein R6 is a hydrogen atom.

In another embodiment, the PPAR agonist is a compound of formula (Ib) wherein R6 is a halogen atom.

In another embodiment, the PPAR agonist is a compound of formula (Ib) wherein R4 and R6 are halogen atoms. In another embodiment, the PPAR agonist is a compound of formula (Ib) wherein R4 and R6 are the halogen atom. In another embodiment, the PPAR agonist is a compound of formula (Ic) wherein R4 and R6 are a chlorine atom In another embodiment, the PPAR agonist is a compound of formula (Ib) wherein R2 is a (C1-C3)alkyl group substituted by a —COOR3 group. In another embodiment, the PPAR agonist is a compound of formula (Ib) wherein R2 is a C(CH₃)₂ group substituted by a —COOR3 group.

In another embodiment, the PPAR agonist is a compound of formula (Ib) wherein R3 is a hydrogen atom or a (C1-C4)alkyl group. In yet another embodiment, R3 is a hydrogen atom.

In another embodiment, the PPAR agonist is a compound of formula (Ib) wherein A is a CH₂—CH₂ group.

In another particular embodiment, the PPAR agonist is a compound of formula (Ib) wherein:
X1 is a R1 group;
R1 is a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 represents a halogen atom;
R5 represents a hydrogen atom;
R6 represents a hydrogen atom;
L2 is a carbonyl group; and
A is a CH₂—CH₂ group.

In another particular embodiment, the PPAR agonist is a compound of formula (Ib) wherein:
X1 is a R1 group;
R1 is a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 represents a halogen atom;
R5 represents a hydrogen atom;
R6 represents a halogen atom;
L2 is a carbonyl group; and
A is a CH₂—CH₂ group.

In another particular embodiment, the PPAR agonist is a compound of formula (Ic) wherein X1 is a R1 group. In another particular embodiment, the PPAR agonist is a compound of formula (Ic) wherein X1 is a R1 group wherein R1 is a (C1-C6)alkyl group substituted by at least one halogen atoms. In another particular embodiment, the PPAR agonist is a compound of formula (Ic) wherein X1 is a R1 group wherein R1 is a CF3 group.

In another particular embodiment, the PPAR agonist is a compound of formula (Ic) wherein L2 represents a —CH—OR7 group.

In another embodiment, the PPAR agonist is a compound of formula (Ic) wherein R7 is an unsubstituted (C1-C4)alkyl group or a (C1-C4)alkyl group substituted by a (C6-C14)aryl group.

In another embodiment, the PPAR agonist is a compound of formula (Ic) wherein R7 is a methyl group or a benzyl group.

In a further particular embodiment, the PPAR agonist is a compound of formula (Ic) wherein R4 is a halogen atom. In yet another embodiment, R4 is a chlorine atom in formula (Ic).

In a further particular embodiment, the PPAR agonist is a compound of formula (Ic) wherein R5 is a hydrogen atom.

In another embodiment, the PPAR agonist is a compound of formula (Ic) wherein R6 is a halogen atom.

In another embodiment, the PPAR agonist is a compound of formula (Ic) wherein R4 and R6 are halogen atoms. In another embodiment, the PPAR agonist is a compound of formula (Ic) wherein R4 and R6 are the halogen atom. In another embodiment, the PPAR agonist is a compound of formula (Ic) wherein R4 and R6 are a chlorine atom

In another embodiment, the PPAR agonist is a compound of formula (Ic) wherein R2 is a (C1-C3)alkyl group substituted by a —COOR3 group. In another embodiment, the PPAR agonist is a compound of formula (Ic) wherein R2 is a C(CH₃)₂ group substituted by a —COOR3 group.

In another embodiment, the PPAR agonist is a compound of formula (Ic) wherein R3 is a hydrogen atom or a (C1-C4)alkyl group. In yet another embodiment, R3 is a hydrogen atom.

In another embodiment, the PPAR agonist is a compound of formula (Ib) wherein A is a CH₂—CH₂ group.

In another particular embodiment, the PPAR agonist is a compound of formula (Ic) wherein:
X1 is a R1 group;
R1 is a (C1-C4)alkyl group substituted by at least one halogen atom;
R2 is a C(CH₃)₂ group substituted by a —COOR3 group;
R3 is a hydrogen atom or an unsubstituted (C1-C4)alkyl group;
R4 represents a halogen atom;
R5 represents a hydrogen atom;
R6 represents a halogen atom;
L2 is a carbonyl group; and
A is a CH₂—CH₂ group.

In a particular embodiment, the compound of formula (I) is selected from:
Cpd.1: 2-[4-(3-methoxy-3-(4-(trifluoromethoxy)phenyl)propyl)-2,6-dimethylphenoxy]-2-methylpropanoic acid or a pharmaceutically acceptable salt thereof;
Cpd.2: 2-[2,6-dimethyl-4-[3-[4-(trifluoromethyloxy)phenyl]-3-oxo-propyl]phenoxy]-2-methylpropanoic acid or a pharmaceutically acceptable salt thereof;
Cpd.3: 2-[2,6-dimethyl-4-[3-[4-(trifluoromethyl)phenyl]-3-oxo-propyl]phenoxy]-2-methylpropanoic acid or a pharmaceutically acceptable salt thereof;
Cpd.4: 2-[2,6-dimethyl-4-[3-[4-(methylthio)phenyl]-3-methoxypropyl]phenoxy]-2-methylpropanoic acid or a pharmaceutically acceptable salt thereof;
Cpd.5: 2-[2,6-dimethyl-4-[3-[4-(methylthio)phenyl]-3-isopropyloxypropyl]phenoxy]-2-methylpropanoic acid or a pharmaceutically acceptable salt thereof;
Cpd.6: 2-(4-(3-hydroxy-3-(4-(trifluoromethoxy)phenyl)propyl)-2,6-dimethylphenoxy)-2-methylpropanoic acid;
Cpd.7: 2-(4-(3-(methoxyimino)-3-(4-(trifluoromethoxy)phenyl)propyl)-2,6-dimethylphenoxy)-2-methylpropanoic acid or a pharmaceutically acceptable salt thereof;
Cpd.8: 2-(2-chloro-4-(3-(4-methyl-2-(4-(trifluoromethyl)phenyl)-thiazol-5-yl)-3-oxopropyl)phenoxy)-2-methylpropanoic acid or a pharmaceutically acceptable salt thereof;
Cpd.9: 2-(2,3-dichloro-4-(3-ethoxy-3-(4-methyl-2-(4-(trifluoromethyl)-phenyl)thiazol-5-yl)propyl)phenoxy)-2-methylpropanoic acid or a pharmaceutically acceptable salt thereof;
Cpd.10: 2-(4-(3-(benzyloxy)-3-(5-(4-(trifluoromethyl)phenyl)thien-2-yl)propyl)-2,3-dichlorophenoxy)-2-methylpropanoic acid or a pharmaceutically acceptable salt thereof;
Cpd.11: 2-(2,3-dichloro-4-(3-methoxy-3-(5-(4-(trifluoromethyl)phenyl)-thien-2-yl)propyl)phenoxy)-2-methylpropanoic acid or a pharmaceutically acceptable salt thereof;
Cpd.13: 2-[2,6-dimethyl-4-[3-[4-(methylthio)phenyl]-3-methoxypropyl]phenoxy]-2-methylpropanoic acid or a pharmaceutically acceptable salt thereof;
Cpd.14: 2-[2,6-dimethyl-4-[3-[4-bromophenyl]-3-methoxypropyl]phenoxy]-2-methylpropanoic acid or a pharmaceutically acceptable salt thereof;
Cpd.17: bezafibrate;
Cpd.18: pemafibrate;
Cpd.19: fenofibrate;
Cpd.20: seladelpar lysine;
Cpd.21: pioglitazone;
Cpd.22: rosiglitazone; and
Cpd.23: lanifibranor.

In a more particular embodiment, the compound of formula (I) is Cpd.1: 2-[4-(3-methoxy-3-(4-(trifluoromethoxy)phenyl)propyl)-2,6-dimethylphenoxy]-2-methylpropanoic acid or a pharmaceutically acceptable salt thereof.

The compound of formula (I) can be in the form of a pharmaceutically acceptable salt, particularly acid or base salts compatible with pharmaceutical use. Salts of compounds of formula (I) include pharmaceutically acceptable acid addition salts, pharmaceutically acceptable base addition salts, pharmaceutically acceptable metal salts, ammonium and alkylated ammonium salts. These salts can be obtained during the final purification step of the compound or by incorporating the salt into the previously purified agonist.

The present invention also relates to a pharmaceutically acceptable salt of 2-[4-(3-methoxy-3-(4-(trifluoromethoxy)phenyl)propyl)-2,6-dimethylphenoxy]-2-methylpropanoic acid. In a particular embodiment, the pharmaceutically acceptable salt of 2-[4-(3-methoxy-3-(4-(trifluoromethoxy)phenyl)propyl)-2,6-dimethylphenoxy]-2-methylpropanoic acid is the sodium, calcium, L-lysine or glycine salt thereof.

In a particular embodiment, the invention relates to the sodium salt of 2-[4-(3-methoxy-3-(4-(trifluoromethoxy)phenyl)propyl)-2,6-dimethylphenoxy]-2-methylpropanoic acid.

In a particular embodiment, the invention relates to the calcium salt of 2-[4-(3-methoxy-3-(4-(trifluoromethoxy)phenyl)propyl)-2,6-dimethylphenoxy]-2-methylpropanoic acid.

In a particular embodiment, the invention relates to the L-lysine salt of 2-[4-(3-methoxy-3-(4-(trifluoromethoxy)phenyl)propyl)-2,6-dimethylphenoxy]-2-methylpropanoic acid.

In a particular embodiment, the invention relates to the glycine salt of 2-[4-(3-methoxy-3-(4-(trifluoromethoxy)phenyl)propyl)-2,6-dimethylphenoxy]-2-methylpropanoic acid.

In another aspect, the invention relates to a PPAR agonist for use in the treatment of liver failure, wherein the PPAR agonist is selected from pharmaceutically acceptable salts of 2-[4-(3-methoxy-3-(4-(trifluoromethoxy)phenyl)propyl)-2,6-dimethylphenoxy]-2-methylpropanoic acid. In a particular embodiment, the PPAR agonist for use according to the invention is the sodium, calcium, L-lysine or glycine salt of 2-[4-(3-methoxy-3-(4-(trifluoromethoxy)phenyl)propyl)-2,6-dimethylphenoxy]-2-methylpropanoic acid.

In another aspect, the present invention relates to a compound selected from
Cpd.13: 2-[2,6-dimethyl-4-[3-[4-(methylthio)phenyl]-3-methoxypropyl]phenoxy]-2-methylpropanoic acid or a pharmaceutically acceptable salt thereof; and
Cpd.14: 2-[2,6-dimethyl-4-[3-[4-bromophenyl]-3-methoxypropyl]phenoxy]-2-methylpropanoic acid or a pharmaceutically acceptable salt thereof.

### Sepsis

As mentioned above, the term “sepsis” as used herein refers to a deleterious systemic inflammatory response to infection, formally defined as the presence of infection together with systemic manifestations of infection. The term sepsis as used herein encompasses sepsis, at any degree of severity, and complications thereof such, such as sepsis with multiple organ failure and septic shock.

In a particular embodiment of the invention, the subject suffers or is at risk of suffering from sepsis or complications thereof.

In another particular embodiment, the subject suffers from sepsis caused by one or more microbial species. In particular, the subject may suffer from sepsis caused by a bacterial, fungal or viral infection. In yet another embodiment, said sepsis is cause by a bacterial infection.

In the context of the present invention, the PPAR agonist is administered to a subject, in a therapeutically effective amount. A “therapeutically effective amount” refers to an amount of the drug effective to achieve a desired therapeutic result. A therapeutically effective amount of a drug may vary according to factors such as the disease state, age, sex, and weight of the individual, and the ability of drug to elicit a desired response in the individual. A therapeutically effective amount is also one in which any toxic or detrimental effects of agent are outweighed by the therapeutically beneficial effects. The effective dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above.

The PPAR agonist can be formulated in a pharmaceutical composition further comprising one or several pharmaceutically acceptable excipients or vehicles (e.g. saline solutions, physiological solutions, isotonic solutions, etc.), compatible with pharmaceutical usage and well-known by one of ordinary skill in the art. These compositions can also further comprise one or several agents or vehicles chosen among dispersants, solubilisers, stabilisers, preservatives, etc. Agents or vehicles useful for these formulations (liquid and/or injectable and/or solid) are particularly methylcellulose, hydroxymethylcellulose, carboxymethylcellulose, polysorbate 80, mannitol, gelatin, lactose, vegetable oils, acacia, liposomes, etc. These compositions can be formulated in the form of injectable suspensions, syrups, gels, oils, ointments, pills, tablets, suppositories, powders, gel caps, capsules, aerosols, etc., eventually by means of galenic forms or devices assuring a prolonged and/or slow release. For this kind of formulations, agents such as cellulose, carbonates or starches can advantageously be used.

The PPAR agonist may be administered by different routes and in different forms. For example, it may be administered via a systemic way, per os, parenterally, by inhalation, by nasal spray, by nasal instillation, or by injection, such as intravenously, by intramuscular route, by subcutaneous route, by transdermal route, by topical route, by intra-arterial route, etc. Of course, the route of administration will be adapted to the form of the drug according to procedures well known by those skilled in the art.

In a particular embodiment, the compound is formulated as a tablet. In another particular embodiment, the compound is administered orally.

The frequency and/or dose relative to the administration can be adapted by one of ordinary skill in the art, in function of the patient, the pathology, the form of administration, etc. Typically, the PPAR agonist can be administered at a dose comprised between 0.01 mg/day to 4000 mg/day, such as from 50 mg/day to 2000 mg/day, such as from 100 mg/day to 2000 mg/day; and particularly from 100 mg/day to 1000 mg/day. Administration can be performed daily or even several times per day, if necessary. In one embodiment, the compound is administered at least once a day, such as once a day, twice a day, or three times a day. In a particular embodiment, the PPAR agonist is administered once or twice a day. In particular, oral administration may be performed once a day, during a meal, for example during breakfast, lunch or dinner, by taking a tablet comprising the PPAR agonist.

Suitably, the course of treatment with the PPAR agonist is for at least 1 week, in particular for at least 2, 3, 4, 5, 6, 7, 8, 9, 10, 15, 20 or 24 weeks or more. In a particular embodiment, the course of treatment is for at least 1 month, at least 2 months or at least 3 months. In a particular embodiment, the course of treatment is for at least 1 year, or more depending on the condition of the subject being treated.

In a particular embodiment, the method of treatment consists of the administration of a PPAR agonist as a single active ingredient.

In another particular embodiment, the administration of the PPAR agonist is performed in combination with another active ingredient, preferably with an antimicrobial agent such as an antibiotic, an antifungal or an antiviral. Of course, the most suitable antimicrobial agent will be selected depending on the organism or virus responsible for the infection, as is well known in the art. In a particular embodiment, the sepsis is caused by a bacterial infection, and the antimicrobial is an antibiotic. Antibiotics useful in the treatment of bacterial infections are well known in the art. Illustrative antibiotic families include, without limitation, beta-lactam antibiotics (such as penicillins), tetracyclines, cephalosporins, quinolones, lincomycins, macrolides, sulfonamides, glycopeptides, aminoglycosides and carbapenems. In a particular embodiment, the PPAR agonist can be combined to an antibiotic of the carbapenem family, such as ertapenem.

The PPAR agonist and the antimicrobial agent can be administered to the subject in the same or separate pharmaceutical compositions. In a particular embodiment, the invention provides a pharmaceutical composition comprising the PPAR agonist, an antimicrobial agent and a pharmaceutically acceptable excipient. This pharmaceutical composition can be used in the method of the invention, for the treatment of sepsis. In another embodiment, the invention provides a method wherein
a first pharmaceutical composition comprising the PPAR agonist and a pharmaceutically acceptable excipient; and
a second pharmaceutical composition comprising the antimicrobial agent; are both administered to the subject for the treatment of sepsis.

The first and second pharmaceutical compositions can be used simultaneously, separately or sequentially (i.e. the first pharmaceutical composition can be administered before or after the second pharmaceutical composition). As such, the invention also provides a kit-of-parts comprising:
a first pharmaceutical composition comprising the PPAR agonist and a pharmaceutically acceptable excipient; and
a second pharmaceutical composition comprising the antimicrobial agent;
for simultaneous, separate or sequential use in the treatment of sepsis.

The following examples serve to illustrate the invention and must not be considered as limiting the scope thereof.

### Examples

### Chemistry

Chemical names follow IUPAC nomenclature. Starting materials and solvents were purchased from commercial suppliers (Acros Organic, Sigma Aldrich, Combi-Blocks, Fluorochem, Fluka, Alfa Aesar or Lancaster) and were used as received without further purification. Some starting materials can be readily synthesized by a person skilled in the art. Air and moisture sensitive reactions were carried out under an inert atmosphere of nitrogen, and glassware was oven-dried. No attempts were made to optimize reaction yields. Chemical shifts (6) are reported in ppm (parts per million), by reference to the hydrogenated residues of deuterated solvent as internal standard: 2.50 ppm for DMSO-d6, 7.26 ppm for CDCl3, and 3.31, and 4.78 for Methanol-d4. The spectral splitting patterns are designated as follows: s, singlet; d, doublet; dd, doublet of doublets; ddd, doublet of doublet of doublets; t, triplet; dt, doublet of triplets; q, quartet; m, multiplet; br s, broad singlet.

### Animal Experimentation

Manipulation of animals was conducted carefully in order to reduce stress at the minimum. All the experiments were performed in compliance with the guidelines of French Ministry of Agriculture for experiments with laboratory animals (law 87-848). The study was conducted in compliance with Animal Health Regulation (Council directive No. 2010/63/UE of Sep. 22, 2010 and French decree no. 2013-118 of Feb. 1, 2013 on protection of animals).

### Example 1: Synthesis of Compounds According to the Invention

Compounds of formula (I) can be synthetized following general procedures disclosed in WO2005005369, WO2007147879, WO2007147880, WO2008087366 and WO2008087367.

### Example 1a: sodium 2-{4-[3-methoxy-3-(4-trifluoromethoxy-phenyl)-propyl]-2,6-dimethyl-phenoxy}-2-methyl-propionate

2-{4-[3-Methoxy-3-(4-trifluoromethoxy-phenyl)-propyl]-2,6-dimethyl-phenoxy}-2-methyl-propionic acid (prepared as disclosed in WO2007147880) (500 mg, 0.001135 mol) and sodium hydroxide (45 mg, 0.0011 mol) were mixed in methanol (10 mL, 0.2 mol) and stirred at 40° C. (rotavap) for 10 minutes before methanol was evaporated to dryness to give sodium 2-{4-[3-methoxy-3-(4-trifluoromethoxy-phenyl)-propyl]-2,6-dimethyl-phenoxy}-2-methyl-propionate (515 mg) as a white solid. mp: 284° C.; 1H NMR (D20,300 MHz, 6 in ppm): 1.1 (s, 6H); 1.55 (m, 1H); 1.7 (m, 1H); 2.0 (s, 6H); 2.1 (m, 1H); 2.2 (m, 1H); 2.8 (s, 3H); 3.8 (t, 1H); 6.5 (s, 2H); 6.95 (m, 4H)—purity (HPLC): 98.7%.

### Example 1b: L-lysine 2-{4-[3-methoxy-3-(4-trifluoromethoxy-phenyl)-propyl]-2,6-dimethyl-phenoxy}-2-methyl-propionate

2-{4-[3-Methoxy-3-(4-trifluoromethoxy-phenyl)-propyl]-2,6-dimethyl-phenoxy}-2-methyl-propionic acid (prepared as disclosed in WO2007147880) (500 mg, 0.001135 mol) and L-Lysine (160 mg, 0.0011 mol) were mixed in methanol (10 mL, 0.2 mol) and stirred at 40° C. (rotavap) for 10 minutes before methanol was evaporated to dryness to give L-lysine 2-{4-[3-methoxy-3-(4-trifluoromethoxy-phenyl)-propyl]-2,6-dimethyl-phenoxy}-2-methyl-propionate (657 mg) as a white solid. mp: 176° C.—1H NMR (D20, 300 MHz, 6 in ppm): 1.15 (s, 6H); 1.1-1.2 (m, 1H); 1.3-1.5 (m, 2H); 1.5-1.7 (m, 5H); 1.7-1.85 (m, 3H); 1.9-2 (m, 1H); 2.0 (s, 6H); 2.05-2.15 (m, 1H); 2.15-2.2.3 (m, 1H); 2.8-2.95 (m, 2H); 2.85 (s, 3H); 3.65 (t, 1H); 3.9 (t, 1H); 6.5 (s, 2H); 6.95 (m, 4H)—purity (HPLC): 100% Other salts of compounds disclosed in WO2007147880 or WO2007147879 can be produced according to the preceding methods.

### Example 1c: 2-(4-(3-methoxy-3-(4-(trifluoromethyl)phenyl)propyl)-2,6-dimethylphenoxy)-2-methylpropanoic acid

2-(4-(3-methoxy-3-(4-(trifluoromethyl)phenyl)propyl)-2,6-dimethylphenoxy)-2-methylpropanoic acid can be prepared as disclosed in WO2007147880.

1H NMR (300 MHz, DMSO d6, 6 in ppm): 1.31 (s, 6H); 1.76-2.00 (m, 2H); 2.10 (s, 6H); 2.36-2.58 (m, 2H); 3.14 (s, 3H); 4.24 (m, 1H); 6.77 (s, 2H); 7.51 (d, 2H, J=8.2 Hz); 7.72 (d, 2H, J=8.2 Hz); 12.77 (br s, 1H)—purity (HPLC): 99.7%—mass: 447 MNa+

### Example 1d: 2-[2,6-dimethyl-4-[3-[4-bromophenyl]-3-methoxypropyl]phenoxy]-2-methylpropanoic acid

2-[2,6-dimethyl-4-[3-[4-bromophenyl]-3-methoxypropyl]phenoxy]-2-methylpropanoic acid can be prepared as disclosed in WO2007147880.

1H NMR (300 MHz, DMSO d6, 6 in ppm): 1.31 (s, 6H); 1.74-1.98 (m, 2H); 2.10 (s, 6H); 2.33-2.43 (m, 2H); 3.10 (s, 3H); 4.11 (t, 1H, J=5.5 Hz); 6.76 (s, 2H); 7.25 (d, 2H, J=8.5 Hz); 7.55 (d, 2H, J=8.1 Hz); 12.79 (br s, 1H)—purity (HPLC): 99%—mass: 457/459 MNa+

### Example 2: The Compounds According to the Invention Inhibit Monocyte Differentiation into Macrophages

In order to test the efficacy of the compounds to inhibit the activation of the immune system, we used the human monocytic cell line THP-1 (Sigma). THP1 monocytes were cultured in RPMI 1640 with L-glutamine medium (#10-040-CV, Corning) supplemented with 10% fetal bovine serum (FBS, #10270, Gibco), 1% penicillin/streptomycin (#15140, Gibco) and 25 mM Hepes (H0887, Sigma) in a 5% C02 incubator at 37° C.

To evaluate the effect of the compounds on monocyte differentiation, 2.5×10⁴ THP-1 cells were cultured for 24 h in a 384-well plate in FBS-deprived culture medium containing Cpd.1 in dose ranges, as well as 5 or 100 ng/mL phorbol 12-myristate 13-acetate (PMA, #P8139, Sigma), as indicated, to induce differentiation into macrophages.

Tumor necrosis a (TNFα) and monocyte chemoattractant protein 1 (MCP1) were measured in cell supernatants by Homogeneous Time Resolved Fluorescence (HTRF, #62HTNFAPEG for TNFα, and 62HCCL2PEG for MCP1, Cisbio). Fluorescence was measured with Infinite 500 (#30019337, Tecan) to determine the concentration of cytokines.

### Results

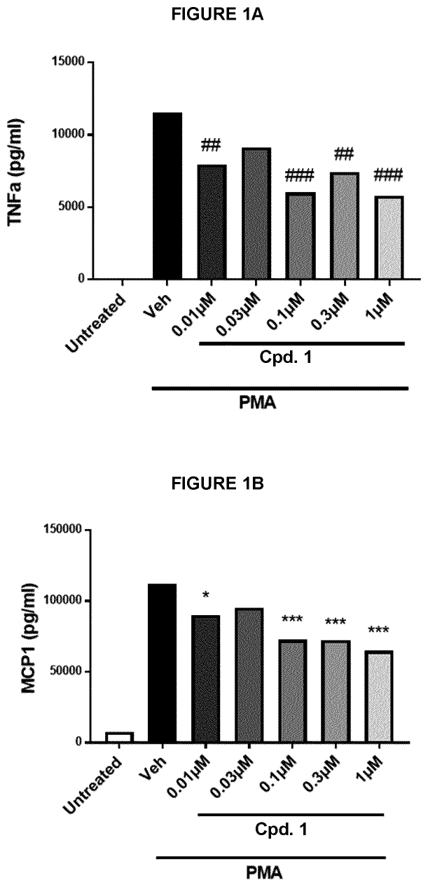

The results are shown in FIGS. 1A and 1B. While PMA induces the secretion of TNFα and MCP1, markers of differentiation of monocytes to macrophages, Cpd.1 drastically reduces TNFα (FIG. 1A) and MCP1 (FIG. 1B) content in the supernatant, reaching circa 50% inhibition at the dose of 1 μM.

Taken together, these results show the efficacy of the compounds according to the invention to reduce the immune system activation.

### Example 3: The Compounds According to the Invention Inhibit Macrophage Activation

In order to test the efficacy of the compounds on macrophage activation and pro-inflammatory cytokine production, 2.5×10⁴ THP-1 cells were cultured in a 384-well plate and treated with 100 ng/mL PMA (#P8139, Sigma) for 24 h to induce differentiation into macrophages.

Then, medium was removed and FBS-deprived medium containing the compound of formula (I) was added for 24 h. Finally, THP1 macrophages were stimulated with 100 ng/mL LPS (E. coli O55:B5, #L4005, Sigma) for 6 h.

### Results

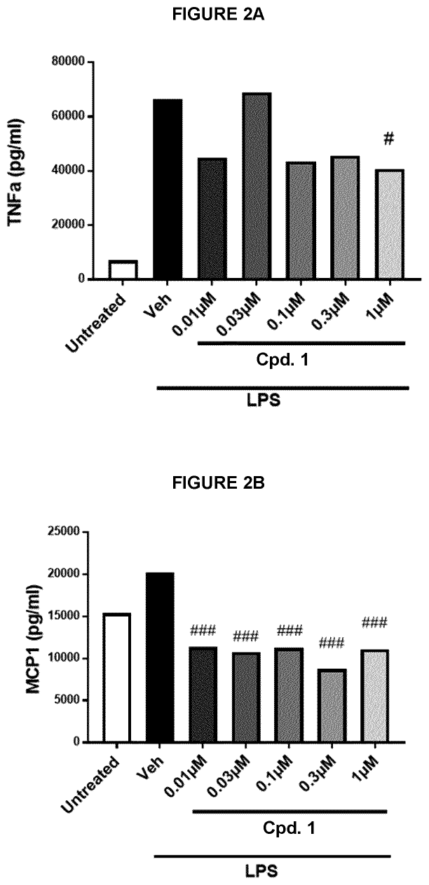

The results are shown in FIG. 2. Cpd.1.3 also potent at reducing TNFα and MCP1 secretion (FIGS. 2A and 2B), overpassing the untreated condition for MCP1 secretion at all doses tested (FIG. 2B).

These results show the potency of the compounds of formula (I) to counteract macrophage activation and limit cytokines production, thereby protecting damages to the tissues.

### Example 4: Compounds According to the Invention Reduce Circulating Cytokine Levels in a Model of Endotoxemia

Endotoxemia occurs in many patients with sepsis and participates to the exacerbation of the host response leading to the septic shock. The activation of immune cells, such as macrophages, by LPS produces inflammatory cytokines that induce parenchymal cell death in different tissues (liver, kidney, etc) which can eventually lead to multiple organ failure.

### Preclinical Model of Endotoxemia

To evaluate the efficacy of the compounds to reduce the cytokine production in response to LPS-induced endotoxemia, male Sprague Dawley rats of 250-275 g from Janvier Labs (France) received a single intraperitoneal injection of 1 mg/kg LPS (Escherichia coli 0111:B4, #L2630, Sigma).

Cpd.1 (3 mg/kg/day) or vehicle (Labrafil M 1944 CS, #3063, Gattefossé) was administered by oral gavage during the 3 days before LPS injection. Rats were euthanized by cervical dislocation 3 hours after treatment. Blood samples were obtained from retro-orbital sinus puncture on animals slightly asleep with isoflurane (Isoflurin 1000 mg/g, GTIN 03760087152678, Axience) just before sacrifice.

The serum concentrations of interleukin-6 (IL6) and interleukin-1β (IL1β) were determined by ELISA (SR6000B and SRLB00, respectively, R&D Systems).

### Results

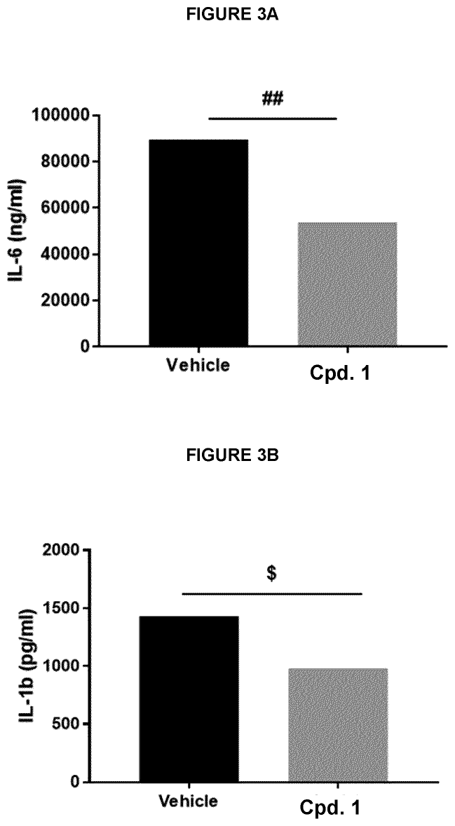

LPS injection induced a high production of the pro-inflammatory cytokines IL6 and IL1β, reaching 80 μg/mL and 1500 pg/mL, respectively, in the sera (undetectable in healthy animals). Cpd.1 drastically reduced IL6 and IL1β levels by 40% and 31%, respectively (FIGS. 3A and 3B).

These results show that treatment with Cpd.3 reduces circulating cytokines levels in response to LPS in vivo, thereby protecting from tissue damages and organ failure.

### Example 5: Compounds According to the Invention Protect from Alterations in Hepatic Function and Systemic Inflammation Induced by Endotoxins

Endotoxin such as lipopolysaccharide (LPS) is recognized as the most potent microbial mediator implicated in the pathogenesis of sepsis and septic shock. Elevation of circulating endotoxins occurs during sepsis and induces alterations in hepatic function, such as hypoalbuminemia, associated with morbidity and mortality (Wang et al. J Surg Res 2004, 121(1), p 20-4; Gatta et al. Intern Emerg Med 2012, Suppl 3, :S193-9).

### Preclinical Model of Endotoxemia

To evaluate the efficacy of the compounds on hepatic markers concentration in response to LPS-induced endotoxemia, male Sprague Dawley rats of 250-275 g from Janvier Labs received a single intraperitoneal injection of 1 mg/kg LPS (Escherichia coli 0111:B4, #L2630, Sigma-Aldrich).

Cpd.1 (3 mg/kg/day), Cpd.19 (100 mg/kg/day) or vehicle (Labrafil M 1944 CS, #3063, Gattefossé for Cpd.1 or carboxymethylcellulose 1%, 0.1% Tween 80 for Cpd.19) was administered by oral gavage during the 3 days before LPS injection. Rats were euthanized by cervical dislocation 3 hours after LPS treatment. Blood samples were obtained from retro-orbital sinus puncture on animals slightly asleep with isoflurane (Isoflurin 1000 mg/g, GTIN 03760087152678, Axience) just before sacrifice.

### Evaluation of Hepatic Markers Concentration in Rat Serum

The serum concentration of total bilirubin was measured using the Randox kit for Daytona plus automate (#BR3859, Randox Laboratories). Briefly, total bilirubin is quantified by a colorimetric 35 assay based on the method described by by Jendrassik L, and Gróf P., Biochem Zeitschrift 1938, 297, p 82-9.

The serum concentration of albumin was measured using the Randox kit for Daytona plus automate (#AB8301, Randox Laboratories). Briefly, the measurement of albumin is based on its quantitative binding to the indicator 3,3′,5,5′-tetrabromo-m cresol sulphonphthalein (bromocresol green). The albumin-BCG-complex absorbs maximally at 578 nm.

### Cytokine Analysis in Rat Serum

The concentration of tumor necrosis a (TNFα) was determined using a multiplex sandwich ELISA system (Rat Premixed Multi-Analyte Kit LXSARM, Biotechne) according to the manufacturer instructions. Briefly, serum samples were added onto magnetic particles pre-coated with cytokines-specific antibodies. After washing, cytokines were detected through the addition of biotinylated antibodies. Finally, streptavidin conjugated with phycoerythrin were added and analysis were carried out with the Luminex 200 analyzer. The signal strength of phycoerythrin is proportional to the concentration of the specific cytokine.

### Results

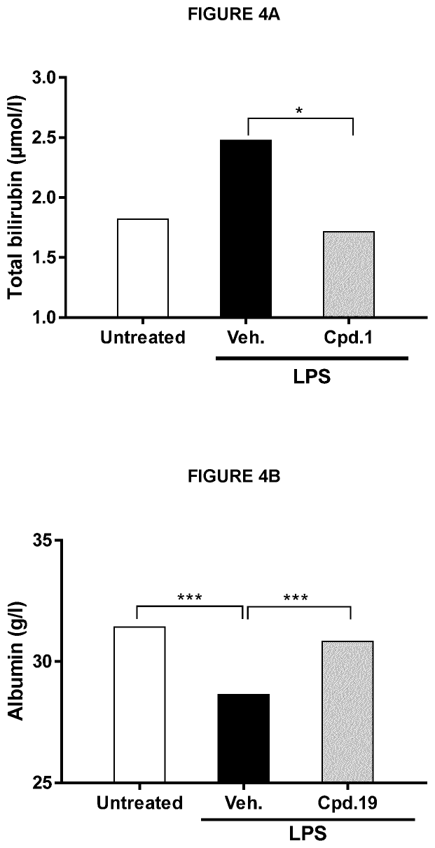

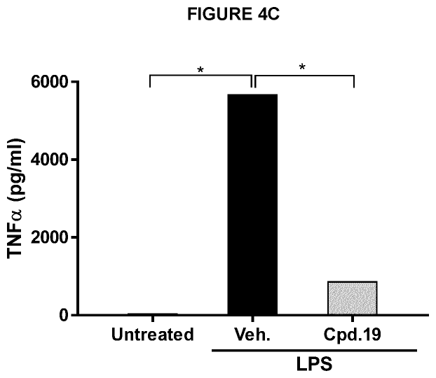

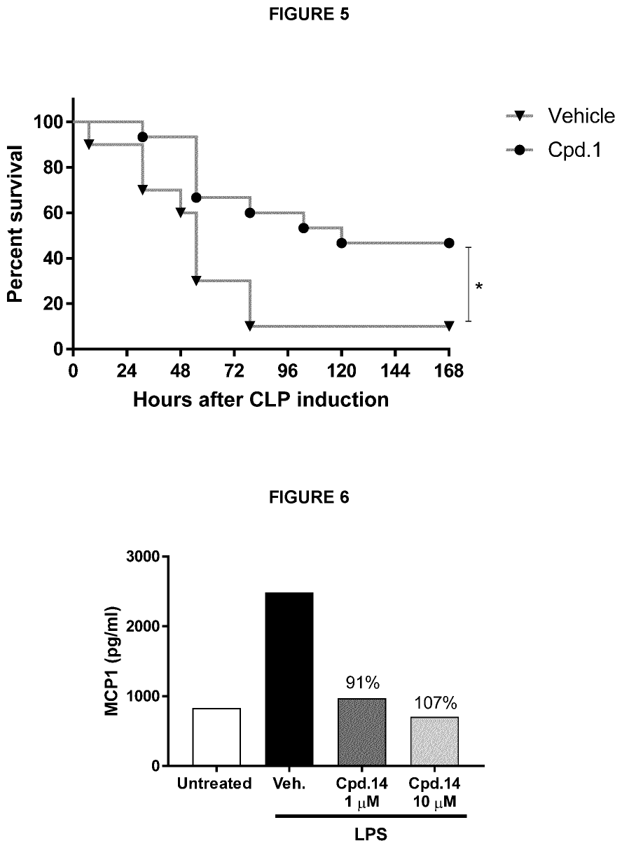

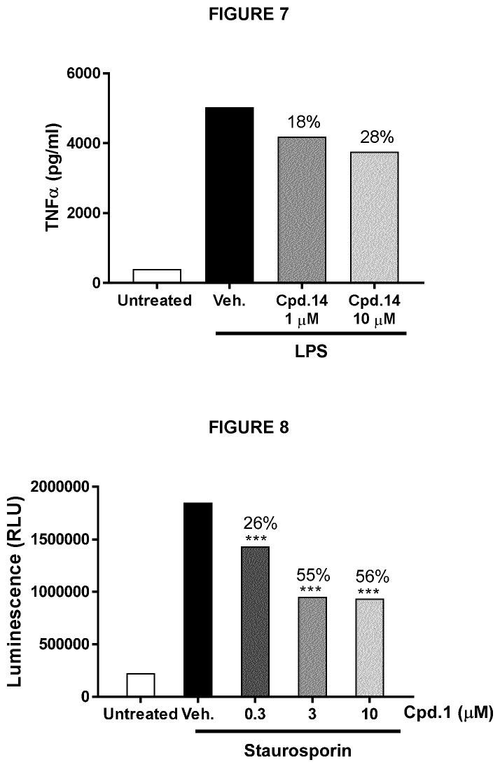

Rats undergoing endotoxemia had altered hepatic function as shown by high total bilirubin concentration in the serum (FIG. 4A). When administrated to rats undergoing endotoxemia, Cpd.1 completely restored the level of total bilirubin, compared to the vehicle condition.

In parallel, while LPS injection led to a decrease of the serum albumin level, animals treated with Cpd.19 showed a restoration of albumin concentration by 79% when compared to the vehicle control rats (FIG. 4B).

Regarding circulating cytokines, we demonstrated that Cpd.19 drastically reduced the LPS-induced serum TNFα level by 85% (FIG. 4C).

These results show that administration of both Cpd. 1 and Cpd.19 allows to improve hepatic function and inflammation in response to LPS in vivo, thereby protecting from tissue damages and organ failure.

### Example 6: Compounds According to the Invention Improves Survival in a Model of Sepsis

### Aim of the Study

ACLF is a rare clinical condition but remains associated with high short-term mortality either during hospitalization stay or shortly after discharge. A consensual paradigm is emerging implying an overactivation of the innate immune system due to translocation of bacterial products like PAMPs (mainly LPS from Gram negative bacteria) with or without living bacteria from the gut. Such an impaired intestinal barrier provokes an exaggerated endotoxemia resulting in an uncontrolled inflammatory storm which can jeopardize minimal functioning of cirrhotic liver and other vital organs like the kidneys, the brain, the coagulation system, the cardiovascular system and/or the respiratory system.

Polymicrobial sepsis induced by cecal ligation and puncture (CLP) is characterized by dysregulated systemic inflammatory responses followed by immunosuppression. The CLP model in mice mimics the progression and features of human sepsis and is thus also useful to determine whether a drug would be useful in the treatment of ACLF in view of the common pathophysiological features of transition from decompensated cirrhosis to ACLF and from sepsis to septic shock.

This study aims to investigate the efficacy of Cpd.1 in cecal ligation and puncture (CLP) model in C57BL6J (BL6) male mice. The efficacy of the test compound was evaluated based on the survival rate of the animals within the study period.

### Cecal Ligature and Puncture Surgery

C57BL6J male mice (supplier Janvier—France) at 9 weeks of age and weighing 23-25 g on arrival were anesthetized with 250 μL of xylazine/ketamine solution (20 mg/100 g body weight) by intraperitoneal route. A 1-1.5 cm abdominal midline incision was made, and the caecum was located and tightly ligated at half the distance between distal pole and the base of the cecum with 4-0 silk suture (mild grade). The caecum was punctured through-and-through once with a 21-gauge needle from mesenteric toward antimesenteric direction after medium ligation. A small amount of stool was extruded to ensure that the wounds were patent. Then the cecum was replaced in its original position within the abdomen, which was closed with sutures and wound clips. Mice were followed for body weight evolution and mortality rate until Day 6.

### Treatment Scheme

Cpd.1 or vehicle (Labrafil M 1944 CS, #3063, Gattefossé) was administrated at 0.3 mg/kg, p.o. for three days before CLP surgery. The day of CLP (day 0), Cpd.1 was administrated 1 h before surgery and pursued daily until day 6. Experiment was terminated at day 7.

### Experimental Groups

Two groups of C57BL6J mice at the age of 9 weeks were treated as described above.
1. BL6 mice CLP (21G needle)+Vehicle (0.3 mL/kg; p.o.) (10 mice)
2. BL6 mice CLP (21 G needle)+Cpd.1 (0.3 mg/kg; p.o.) (15 mice)

### Clinical Signs

No side effect of Cpd.1 was recorded after each administration. Body weight and survival rate were measured for 7 days.

### Survival Rates Results

The “CLP+Vehicle (p.o.)” group reached 30% of survival rate at Day 2 and 10% at Day 7, until the end of the experiment.
The “CLP+Cpd.1 (0.3 mg/kg, p.o.)” group showed a significant increase in survival rate by 47% at the end of the experiment (Day 7) compared to the “CLP+Vehicle (p.o.)” control group (FIG. 5).

### Conclusion

Cpd.1 (0.3 mg/kg, p.o.) given 3 days before surgery, 1 h before surgery and once daily until Day 7, significantly improved the survival rate in comparison with CLP+vehicle control group.

In conclusion, Cpd.1 has a beneficial effect on survival rate in CLP induced polymicrobial sepsis in mice.

### Example 7: Compounds According to the Invention Inhibit Macrophage Activation

In order to test the efficacy of the compounds on macrophage activation and pro-inflammatory cytokine production, 2.5×10⁴ THP-1 cells were cultured in a 384-well plate and treated with 100 ng/mL PMA (#P8139, Sigma) for 24 h to induce differentiation into macrophages.

Then, medium was removed and FBS-deprived medium containing 1 or 10 μM compound was added for 24 h. Finally, THP1 macrophages were stimulated for 6 h with 100 ng/mL LPS (Klebsiella pneumoniae, #L4268, Sigma-Aldrich).

Monocyte chemoattractant protein 1 (MCP1) and Tumor necrosis a (TNFα) were measured in cell supernatants by Homogeneous Time Resolved Fluorescence (HTRF, #62HTNFAPEG for TNFα, and 62HCCL2PEG for MCP1, Cisbio). Fluorescence was measured with Infinite 500 (#30019337, Tecan) to determine the concentration of cytokines.

### Results

Treatment of macrophages with LPS from Klebsiella led to a 3,5-fold and 13-fold increase of MCP1 and TNFα serum levels, respectively (FIGS. 6 and 7). In order to evaluate the effect of the compounds on LPS-induced cytokines production by macrophages, the percentage of inhibition over the vehicle condition were calculated for each compound at 1 and 10 μM and are presented in Table 1 for MCP1 and Table 2 for TNFα. The results obtained for Cpd.14 are shown through graphs as representative experiments (FIGS. 6 and 7).

TABLE 1

% inhibition of MCP1 secretion over mean Veh.

| Compound | 1 μM | 10 μM |
|---|---|---|
| Cpd. 1 | 108 | 120 |
| Cpd. 2 | 96 | 106 |
| Cpd. 3 | 90 | 107 |
| Cpd. 4 | 100 | 110 |
| Cpd. 5 | 93 | 110 |
| Cpd. 6 | 94 | 103 |
| Cpd. 7 | 95 | 107 |
| Cpd. 9 | 86 | 127 |
| Cpd. 13 | 99 | 111 |
| Cpd. 14 | 91 | 107 |
| Cpd. 17 | 22 | 54 |
| Cpd. 18 | 93 | 123 |
| Cpd. 19 | 24 | 89 |
| Cpd. 20 | 105 | 120 |
| Cpd. 21 | 80 | 121 |
| Cpd. 22 | 79 | 117 |
| Cpd. 23 | 100 | 126 |

TABLE 2

% inhibition of TNFα secretion over mean Veh.

| Compound | 1 μM | 10 μM |
|---|---|---|
| Cpd. 3 | 0 | 20 |
| Cpd. 7 | 25 | 42 |
| Cpd. 9 | 3 | 77 |
| Cpd. 13 | 0 | 18 |
| Cpd. 14 | 18 | 28 |
| Cpd. 21 | 0 | 19 |
| Cpd. 22 | 36 | 11 |
| Cpd. 23 | 15 | 32 |

As shown in example 3, Cpd.1 reduced the production of MCP1 induced by LPS Klebsiella, overpassing the untreated condition for MCP1 secretion (Table 1). Likewise, treatment with 10 μM of Cpd.2, Cpd.3, Cpd.4, Cpd.5, Cpd.6, Cpd.7, Cpd.9, Cpd.13, Cpd.14, Cpd.17, Cpd.18, Cpd.19, Cpd. 20, Cpd.21, Cpd.22 and Cpd.23 decreased MCP1 secretion from 54 to 132% (Table 1).

In parallel, when added at 10 μM, Cpd.3, Cpd.7, Cpd.9, Cpd.13, Cpd.14, Cpd.21, Cpd.22 and Cpd.23 also reduced TNFα secretion by macrophages from 11 to 58% (Table 2).

These results show the potency of the compounds to counteract macrophage activation and limit cytokines production, thereby protecting damages to the tissues.

### Example 8: Compounds According to the Invention Protect Hepatocyte from Apoptosis

In order to evaluate the effect of the compounds on human hepatocytes that undergo a cellular stress induced by staurosporin, the human hepatoblastoma-derived HepG₂ cell line (ECACC, #85011430, Sigma-Aldrich) was cultured in high-glucose DMEM medium (#41965, Gibco, France) supplemented with 10% of fetal bovine serum (FBS, #10270, Gibco), 1% penicillin/streptomycin (#15140, Gibco), 1% sodium pyruvate (#11360, Gibco) and 1% MEM non-essential amino acids (#11140, Gibco) in a 5% CO₂ incubator at 37° C.

To evaluate caspase 3/7 activity, which is a surrogate marker of apoptosis, 1.5×10⁴ cells were plated in a 384-well plate (#781080, Greiner, France). After cell adherence (8 hours), cells were serum starved for 16 h in the presence of 0.3 μM of compounds or vehicle. Cpd.1 was also used at 3 and 10 μM. Thereafter, cells were treated with 10 μM staurosporin (#569397, Sigma-Aldrich, Germany) supplemented with compound for additional 4 hours before cell lysis and caspase activity measurement.

Caspase 3/7 activity was measured using Caspase Glow™ 3/7 assay (#G8093, Promega, USA). Luminescence was measured using a Spark microplate reader (#30086376, Tecan, USA). The amount of luminescence (RLU) directly correlates with caspase 3/7 activity.

### Results

Incubation of HepG2 cells with staurosporin induced apoptosis, as shown by an increase of caspase 3/7 activity by 8-fold (FIG. 8). In order to evaluate the effect of the compounds on staurosporin-induced caspase activity in HepG2 cells, the percentage of inhibition over the vehicle condition were calculated. Interestingly, the addition of Cpd.1 reduced caspase activity induced by staurosporin in a dose-dependent manner (FIG. 8).

Likewise, treatment with 0.3 μM of Cpd.2, Cpd.4, Cpd.6, Cpd.7, Cpd.13, Cpd.14 or Cpd.21 also decreased staurosporin-induced caspase activity from 26 to 79% (Table 3).

TABLE 3

| | % Inhibition of caspase activity over mean Veh. |
|---|---|
| Compound | 0.3 μM |
| Cpd. 1 | 26 |
| Cpd. 2 | 70 |
| Cpd. 4 | 32 |
| Cpd. 6 | 43 |
| Cpd. 7 | 66 |
| Cpd. 13 | 79 |
| Cpd. 14 | 77 |
| Cpd. 21 | 60 |

These results show that the compounds directly protect hepatocyte from cell death by inhibiting caspase activity.

### Conclusion

Altogether, these results show that treatment with compounds according to the invention reduce overt activation of the immune system via direct anti-inflammatory effects on monocytes and macrophages while they also directly reduce apoptosis and protect from alterations of liver functions induced by endotoxins. Therefore, the compounds according to the invention protect from tissue damages, organ failure and death that occur during sepsis and septic shock.

1. A method for the treatment of sepsis in a subject in need thereof, comprising the step of administering to the subject an effective amount of a PPAR agonist, wherein said PPAR agonist is selected from:
lanifibranor, bezafibrate, fenofibrate, pemafibrate, seladelpar, saroglitazar, pioglitazone and rosiglitazone; or
a compound of formula (I), or a pharmaceutically acceptable salt thereof, 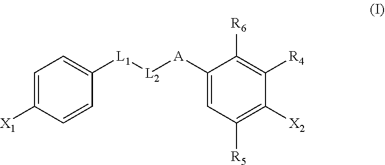
wherein:
X1 represents a halogen atom, a R1 group or a G1-R1 group;
L1 represents a bond, a thiophenyl group or a thiazole group substituted or not by a (C1-C3)alkyl group;
L2 represents:
(i) a —CH—OR7 group, in which R7 represents a hydrogen atom, an unsubstituted (C1-C6)alkyl group or a (C1-C6)alkyl group substituted by a (C6-C14)aryl group;
(ii) a carbonyl group (CO); or
(iii) a C═N—OR8, in which R8 represents an unsubstituted (C1-C6)alkyl group;
A represents a CH═CH or a CH₂—CH₂ group;
X2 represents a G2-R2 group;
G1 and G2, identical or different, represent an atom of oxygen or sulfur;
R1 represents a hydrogen atom, an unsubstituted (C1-C6)alkyl group, a (C6-C14)aryl group or an alkyl group that is substituted by at least one substituent selected from halogen atoms, (C1-C6)alkoxy groups, (C1-C6)alkylthio groups, (C5-C10)cycloalkyl groups, (C5-C10)cycloalkylthio groups and 5- to 14-membered heterocyclic groups;
R2 represents a (C1-C6)alkyl group substituted by a —COOR3 group;
R3 represents a hydrogen atom or a (C1-C6)alkyl group that is substituted or not by at least one substituent selected from halogen atoms, (C5-C10)cycloalkyl groups and 5- to 14-membered heterocyclic groups;
R4 represents a halogen atom, an unsubstituted (C1-C6)alkyl group or a (C1-C6)alkyl group that is substituted by at least one substituent selected from halogen atoms, (C5-C10)cycloalkyl groups and 5- to 14-membered heterocyclic groups;
R5 represents a hydrogen atom, a halogen atom, an unsubstituted (C1-C6)alkyl group or a (C1-C6)alkyl group that is substituted by at least one substituent selected from halogen atoms, (C5-C10)cycloalkyl groups and 5- to 14-membered heterocyclic groups; and
R6 represents a hydrogen atom or a halogen atom
with the proviso that said compound of formula (I) is not:
elafibranor or a pharmaceutically acceptable salt thereof, or
2-[2,6-dimethyl-4-[3-[4-(methylthio)phenyl]-3-oxo-propyl]phenoxy]-2-methylpropanoic acid or a pharmaceutically acceptable salt thereof.

2. The method according to claim 1, wherein said PPAR agonist is a compound of formula (I) wherein X1 is a G1-R1 group and G1 is an oxygen atom.

3. The method according to claim 1, wherein said PPAR agonist is a compound of formula (I) wherein:
G1 is an oxygen atom;
R1 is an unsubstituted (C1-C4)alkyl group or a (C1-C4)alkyl group substituted by at least one halogen atom;
R2 is a (C1-C3)alkyl group substituted by a —COOR3 group;
R3 is a hydrogen atom or an unsubstituted (C1-C4)alkyl group;
R4 and R5 represent a (C1-C4)alkyl group; and
L2 is a —CH—OR7 group.

4. The method according to claim 1, wherein said compound is 2-[4-(3-methoxy-3-(4-(trifluoromethoxy)phenyl)propyl)-2,6-dimethylphenoxy]-2-methylpropanoic acid or a pharmaceutically acceptable salt thereof, 2-[2,6-dimethyl-4-[3-[4-(trifluoromethyloxy)phenyl]-3-oxo-propyl]phenoxy]-2-methylpropanoic acid or a pharmaceutically acceptable salt thereof; 2-[2,6-dimethyl-4-[3-[4-(trifluoromethyl)phenyl]-3-oxo-propyl]phenoxy]-2-methylpropanoic acid or a pharmaceutically acceptable salt thereof; 2-[2,6-dimethyl-4-[3-[4-(methylthio)phenyl]-3-methoxypropyl]phenoxy]-2-methylpropanoic acid or a pharmaceutically acceptable salt thereof; 2-[2,6-dimethyl-4-[3-[4-(methylthio)phenyl]-3-isopropyloxypropyl]phenoxy]-2-methylpropanoic acid or a pharmaceutically acceptable salt thereof; 2-(4-(3-hydroxy-3-(4-(trifluoromethoxy)phenyl)propyl)-2,6-dimethylphenoxy)-2-methylpropanoic acid or a pharmaceutically acceptable salt thereof; 2-(4-(3-(methoxyimino)-3-(4-(trifluoromethoxy)phenyl)propyl)-2,6-dimethylphenoxy)-2-methylpropanoic acid or a pharmaceutically acceptable salt thereof, 2-(2-chloro-4-(3-(4-methyl-2-(4-(trifluoromethyl)phenyl)-thiazol-5-yl)-3-oxopropyl)phenoxy)-2-methylpropanoic acid or a pharmaceutically acceptable salt thereof; 2-(2,3-dichloro-4-(3-ethoxy-3-(4-methyl-2-(4-(trifluoromethyl)-phenyl)thiazol-5-yl)propyl)phenoxy)-2-methylpropanoic acid or a pharmaceutically acceptable salt thereof; 2-(4-(3-(benzyloxy)-3-(5-(4-(trifluoromethyl)phenyl)thien-2-yl)propyl)-2,3-dichlorophenoxy)-2-methylpropanoic acid or a pharmaceutically acceptable salt thereof, 2-(2,3-dichloro-4-(3-methoxy-3-(5-(4-(trifluoromethyl)phenyl)-thien-2-yl)propyl)phenoxy)-2-methylpropanoic acid or a pharmaceutically acceptable salt thereof, 2-(4-(3-methoxy-3-(4-(trifluoromethyl)phenyl)propyl)-2,6-dimethylphenoxy)-2-methylpropanoic acid or a pharmaceutically acceptable salt thereof; or 2-[2,6-dimethyl-4-[3-[4-bromophenyl]-3-methoxypropyl]phenoxy]-2-methylpropanoic acid or a pharmaceutically acceptable salt thereof.

5. The method according to claim 4, wherein said compound is 2-[4-(3-methoxy-3-(4-(trifluoromethoxy)phenyl)propyl)-2,6-dimethylphenoxy]-2-methylpropanoic acid or a pharmaceutically acceptable salt thereof.

6. The method according to claim 1, wherein said subject suffers from or is at risk of sepsis with multiple organ failure.

7. The method according to claim 1, wherein said subject suffers from or is at risk of septic shock.

8. The method according to claim 1, wherein said PPAR agonist is for use as a single active agent in said method.

9. The method according to claim 1, wherein said PPAR agonist is for use in combination with an antimicrobial agent in said method.

10. The method according to claim 9, wherein said antimicrobial agent is an antibiotic.

11. The method according to claim 9, wherein said antimicrobial agent is a carbapenem antibiotic.

12. The method according to claim 9, wherein said antimicrobial agent is ertapenem.
